(19) United States
(12) Patent Application Publication
(10) Pub. No.: US 2010/0130960 A1
(43) Pub. Date: May 27, 2010

(54) Method for intranasal administration of a pharmaceutical composition

(75) Inventor: Mark Francis Spire, Manhattan, KS (US)

Correspondence Address: Intervet/Schering-Plough Animal Health, Patent Dept. K-6-1, 1990, 2000 Galloping Hill Road, Kenilworth, NJ, 07033-0530, US

(73) Assignees: SCHERING-PLOUGH ANIMAL HEALTH CORPORATION, Summit, NJ (US); SCHERING-PLOUGH LTD., Lucerne (CH); SCHERING-PLOUGH PTY. LIMITED, New South Wales (AU)

(21) Appl. No.: 12/327,897

(22) Filed: Dec. 4, 2008

(30) Foreign Application Priority Data
Nov. 25, 2008 (EP) PCT/EP2008/066158

(51) Int. Cl. A61M 31/00

(52) U.S. Cl. 604/514; 604/275

(57) ABSTRACT

Please insert the attached Abstract provided herewith on a separate sheet after the claims. The present invention pertains to a method for administering a predetermined amount of a pharmaceutical composition to an animal comprising the steps of taking a cannula suitable for insertion into a nasal cavity of the animal, slidably connecting the cannula to a delivery device which is suitable for holding the pharmaceutical composition, inserting the cannula via a nostril into the nasal cavity such that a peripheral line of the cannula is contiguous with a circumferential line of the nostril, actuating the delivery device to force the said amount of the pharmaceutical composition through the cannula into the nasal cavity, retracting the cannula from the nasal cavity and removing the cannula from the device. The invention also pertains to an apparatus for performing this method, a cannula for use with the apparatus and a kit containing the required components to perform the method.

### FIELD OF THE INVENTION

The present invention pertains to method for administering a predetermined amount of a pharmaceutical composition into a nasal cavity of an animal, an apparatus suitable for performing this method, a cannula for use with the device and a kit containing components to assemble the apparatus.

### PRIOR ART

Nasal administration of pharmaceutical compositions is commonly known in the art of veterinary medicine. Such compositions may for example be drugs to cure a disease or provide relief, but may also be vaccines to prevent a disease or at least mitigate the clinical signs of a disease. Often, but not necessarily, such compositions have a liquid carrier. As opposed to administration to humans, there are however some typical problems when intra-nasally administering a pharmaceutical composition to an animal. Often big herds of animals need to be treated which makes the amount of time available per animal extremely short, apart from the problem that the animals are often not fixed at a specific locus (as compared to humans sifting for example in a chair), but move around in a stable, corral, meadow etc. Next to this, animal behaviour is far less predictable than human behaviour. One simply cannot expect an animal, for example an animal belonging to any of the families of Bovidae, Suidae, Equidae, Canidae or Felidae, to simply hold its head still such that the composition can be neatly administrated. Indeed, an animal shaking its head is a quite common phenomenon upon noticing the unnatural feeling of a device entering its nasal cavity. It is in particular for this reason that devices are being used which provide as little contact as possible with the nostril or internal walls of the nasal cavity. Commonly a small tube is being used, typically fixed to a dispensing device, which tube is inserted into the nasal cavity upon which a predetermined amount of the pharmaceutical composition is dispensed and delivered into the nasal cavity. Such a device for example is the PUMP-IT™ Vaccinator, available from Genesis Instruments, USA. However, with the prior art methods and devices some serious disadvantages are encountered. Often it appears that a substantial amount of the composition does not reach the nasal mucosa, but simply leaks out of the nose. There are several reasons for this. Despite the little contact between the device and the animal's tissue, there is still a considerable amount of uncontrolled movements of the animals which makes delivery complicated. Also, in particular for liquid compositions that are sprayed into the nasal passages in the form of a mist of liquid particles, it appears that the particles regroup and simply leak out of the nose as a fluid. Also sneezing is seen a lot, which sneezing inherently leads to a loss of composition from the nasal cavity. It is commonly acknowledged that such problems could be mitigated by even further reducing the contact between the device and the animal during the administration but this makes application practically almost impossible.

### SUMMARY OF THE INVENTION

The object of the current invention is to overcome or at least mitigate the disadvantages of prior art intra-nasal delivery methods and devices. To this end a method has been devised comprising the steps of taking a cannula suitable for insertion into a nasal cavity of the animal, slidably connecting the cannula to a delivery device which is suitable for holding the pharmaceutical composition, inserting the cannula via a nostril into the nasal cavity such that a peripheral line of the cannula is contiguous with a circumferential line of the nostril, actuating the delivery device to force the said amount of the pharmaceutical composition through the cannula into the nasal cavity, retracting the cannula from the nasal cavity, and removing the cannula from the device.

Surprisingly, applicant found that when using a cannula (i.e. an elongated body suitable for insertion into the nasal cavity and having an internal channel for delivery of a pharmaceutical composition) that snuggly fits in the nostril (also called nare), which cannula is actually pushed against the nostril (in fact the cartilage at the nostril will substantially counteract the pushing force), such that the nostril is substantially closed (i.e. a peripheral line of the cannula is contiguous—in contact—with a circumferential line of the nostril), the object of the invention is being met. This seems to contradict the general belief that one should avoid contact with the animal's nose as much as reasonably possible. However, applicant that has found that when the person that administers the composition to the animal is allowed to push the device into the nose, the amount of operating time needed is significantly decreased. This appears to reduce distress for the animal and thus, decreases the occurrence of uncontrolled movements of the animal. Moreover, in particular when administering fluid compositions, it appears that a better spraying delivery into the nasal cavity is provided which becomes apparent by the fact that substantially less of the composition appears to leak out of the nose after administration. Surprisingly also, sneezing occurs less despite the more intimate contact between the delivery device and the animal when applying the current method. Another feature of the present invention is that the cannula is slidably connected to the device. This means that an operative connection between the cannula and the device is arrived at by performing a sliding relative movement between the cannula and the device, the operative connection needing no more than mere friction to be established (although some additional means such as cooperating notches and indentations or other means may be used). Since the cannula is actually pushed into the nose, applicant recognised that there is a risk that the complete apparatus is pulled out of the operators hand or even breaks when there is a sudden movement of the animals head while the cannula firmly sticks into the animal's nose. By having a slidable connection between the cannula and the actual dispensing device this risk is significantly diminished: upon a sudden movement of sufficient magnitude, the device can simply slide off the cannula (or the cannula can slide off the device, depending on how the actual connection is constituted) to insure mechanical disconnection between the animal and the device in such uncontrolled situation. Next to this, an important advantage of a slidable connection is that easy removal of the cannula, for example to enable replacement of the cannula for a new one, is allowed. It is noted that the steps of the present method need not be taken in the order as presented here-above. For example, although not preferred, one could start with inserting the cannula in the nostril and then slidably connect the device to the cannula.

Also, after administration one could firstly remove the device from the cannula and thereafter retract the cannula from the nasal cavity. Such and other embodiments still fall within the scope of the present invention and the appended claims.

In an embodiment of the invention the delivery device has a hollow protrusion with an outer shape that matches the inner shape of a transport channel of the cannula, which enables sliding the transport channel over the protrusion to form the connection between the cannula and the device. The advantage of this embodiment is that a standard dispensing device (e.g. the PUMP-IT™ Vaccinator as commercially available) can be used since the output channel in the protrusion of the device remains unaltered. Would the cannula be put into the output channel of the device, this would need a specially adapted bigger bored output channel. In a further embodiment the shapes are substantially circular, which greatly enhances the ease at which the cannula can be slided over the protrusion.

The invention also pertains to an apparatus suitable for performing the method of the present invention, comprising a device with a dispensing mechanism for dispensing the said predetermined amount of the composition, a space for holding the pharmaceutical composition, and the cannula, slidably connected to the device.

In an embodiment of the invention the cannula comprises a cylindrical shaped body (i.e. a body being elongated and having at least partly a convex circumference; typically the body is somewhat tapered to form a cone; the body may be truncated, e.g. partly flattened etc.) having a substantially closed internal channel with an opening for supplying the composition to the channel and a nozzle for delivering the composition into the nasal cavity, the body being provided with a radially extending flange that is inclined with respect to the longitudinal axis of the body. The flange serves to form the contact between the cannula and the nostril such that a peripheral line of the cannula is contiguous with a circumferential line of the nostril, thereby substantially closing the nostril. The cannula can be made of any material, for example a plastic such as polypropylene or polystyrene, or even a biodegradable material, a rubber etc.

In an embodiment the flange is inclined over an angle that is at least 30° (but smaller than 90°). It appears that this serves to minimize stress for the animal. Apparently, an angle greater than 30° serves to give a nice fit with a nostril and at the same time prevents the elongated body from being inserted to deep into the nasal cavity. Preferably, the angle is between 30° and 60°, in particular between 40° and 50° to provide on the one hand a comfortable fit and on the other hand make the cannula, even when having only one size available, fit for significantly varying sizes of nostrils.

In an embodiment of the invention the flange is provided with a rim that extends in a plane that is substantially perpendicular to the longitudinal axis of the body. Such a rim (i.e a portion that extends from the main body) may serve to grab the cannula and retract it from the nose when the device and cannula have been separated, e.g. due to an uncontrolled movement of the animal when administrating the pharmaceutical composition. Next to this, the rim may prevent that the cannula enters a nasal cavity completely in case a subject animal appears to have a particularly large nasal opening.

In an embodiment the nozzle is slotted. Such a slot enables to spread the composition in a more uniform way to a wider portion of the mucosa of the nasal cavity.

The invention also pertains to the cannula as such and to a kit containing a device with a dispensing mechanism for administering a predetermined amount of a pharmaceutical composition to an animal, a container having therein the pharmaceutical composition and multiple cannulas. With such a kit, for example a box or other container having therein the components (dispensing device, pharmaceutical compositions, multiple cannulas) gathered by the end user e.g. by ordering the different components at various suppliers, multiple animals can be treated, while in between each or several animals treated, a used cannula can be replaced with a new one to prevent cross-contamination between animals.

### BRIEF DESCRIPTION OF THE FIGURES

The invention will be illustrated referring to the examples of a particular embodiment, not having limiting character per se, in the appended figures:

FIG. 1 is a schematic representation of a device for dispensing a predetermined amount of a pharmaceutical composition.

FIG. 2 is a schematic representation of a side view of a cannula suitable for insertion into a nasal cavity of an animal.

FIG. 3 is a schematic representation of the same cannula as depicted in FIG. 2, but from a back side.

FIG. 4 is a front view of the cannula.

FIG. 5 is a schematic representation of an apparatus for administering a predetermined amount of a pharmaceutical composition into a nasal cavity of an animal, assembled by slidably connecting the cannula of FIG. 2 with the dispensing device of FIG. 1.

FIG. 6 is a schematic representation of the apparatus of FIG. 5, when having the cannula inserted into the nasal cavity of a calf.

FIG. 7 is a detailed view of the cannula as inserted at the nostril, showing a peripheral line of the cannula being contiguous with a circumferential line of the nostril.

### DETAILED DESCRIPTION OF THE INVENTION

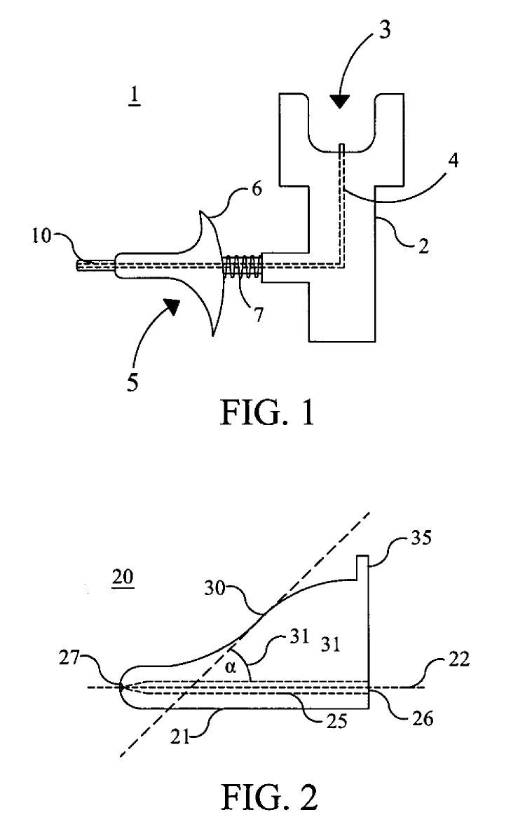

FIG. 1 shows a schematic representation of a device for dispensing a predetermined amount of a pharmaceutical composition. Such a device is for example available from Genesis Instruments under the tradename PUMP-IT™ VACCINATOR. The device 1 comprises a body 2, made of transparent plastic, and having a space 3 for receiving a bottle having therein a pharmaceutical composition. Such composition may for example be the Onset™ 5 IN vaccine available from Intervet/Schering-Plough Animal Health, Kenilworth, USA, or Bovilis IBR Marker vaccine, available from Intervet/Schering-Plough Animal Health, Boxmeer, The Netherlands. The device 1 contains a transport channel 4, that extends between the space 3 and a hollow protrusion 10. This channel serves to provide a transport path for the pharmaceutical composition from the container (not shown) to the outlet of protrusion 10. The device has a dispensing mechanism 5, including a handle 6 and retainment spring 7. The dispensing mechanism can be actuated by retracting the handle 6 towards the body 2, whereupon a predetermined amount of the pharmaceutical composition is expelled from the device through protrusion 10. The spring thereafter pushes the handle back into its original position.

FIG. 2 is a schematic representation of a side view of a cannula 20 suitable for insertion into a nasal cavity of an animal. The cannula comprises a cylindrical shaped body 21, made of a thermformable plastic which retains some elasticity after it has been formed. The body has a substantially closed internal channel 25 with a circular opening 26 which fits over the end of the circular shaped protrusion 10 of device 1 (FIG. 1). The internal channel 25 has a nozzle 27 at an end portion of the body 21. The body is provided with a radially extending flange portion 30, which is inclined with respect to the longitudinal axis 22 of the cylindrical body over an angle a, indicated with numeral 31 in FIG. 2. In this embodiment, the angle is about 45°. This provides the cannula with an excellent fit for nostrils of various sizes. The flange is provided with a rim 35 that can serve as a gripping element when the cannula has to be pulled out of a nostril. Also, it may prevent entrance in a nostril. In this embodiment the cannula has a substantial flattened bottom to fit the substantially flat floor of the nasal passage of a calf. Indeed, for other animals not having such a flat floor in the nasal passage, a flange that extends radially around the entire circumference of the elongated body of the cannula may be better suited.

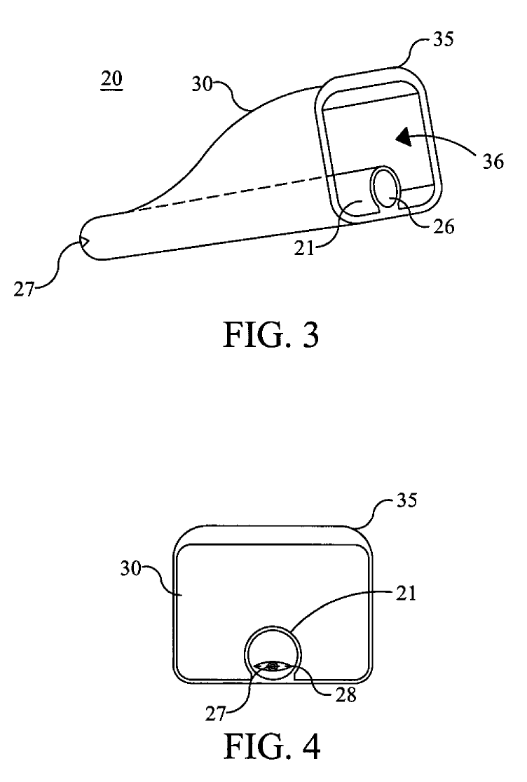

FIG. 3 is a schematic representation of the same cannula as depicted in FIG. 2, but from a back side. From this figure it becomes clear that flange 30 in this particular embodiment encloses a hollow space 36 around elongated cylindrical body 21. This allows the cannula to even better fit various forms of nostrils since the thin walled flange can more easily adapt its shape to a nostril. The shape of the inlet 26 of the elongated body is substantially circular to match the shape of the protrusion 10 of the device 1.

FIG. 4 is a front view of the cannula. In this front view the nozzle 27 can be clearly seen. Also visible is the slot (groove) 28, which serves to ensure a wide spraying angle for the composition.

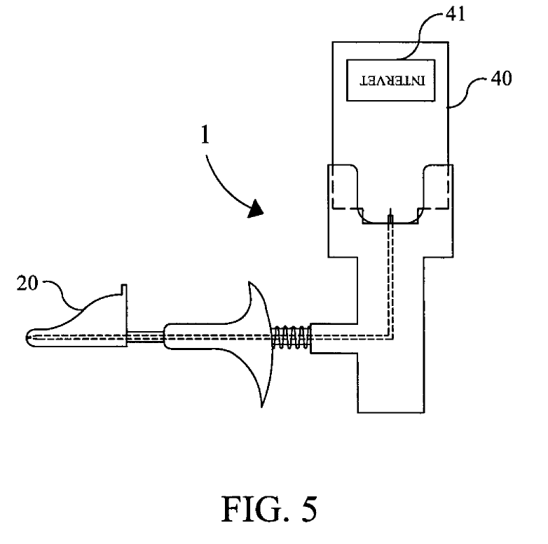

FIG. 5 is a schematic representation of an apparatus for administering a predetermined amount of a pharmaceutical composition into a nasal cavity of an animal, assembled by slidably connecting the cannula of FIG. 2 with the dispensing device of FIG. 1. In this figure the container 40 (with a label 41) having therein the pharmaceutical composition is depicted.

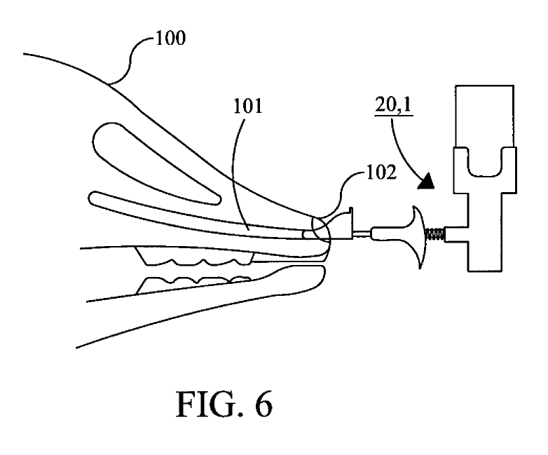

FIG. 6 is a schematic representation of the apparatus of FIG. 5, when having the cannula inserted into the nasal cavity of a calf 100. The cannula has been inserted in the nasal cavity 101, via the nostril 102. The cannula snuggly fits the nostril and the cartilage in the nose around the nostril withstands the force which is being used to push the cannula into the nasal cavity.

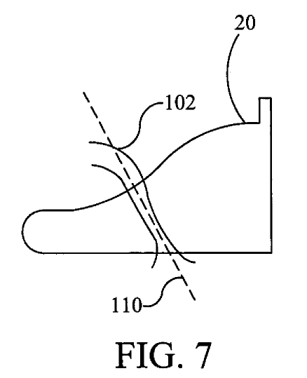

FIG. 7 is a detailed view of the cannula 20 as inserted at the nostril 102, showing a peripheral line of the cannula being contiguous with a circumferential line of the nostril. Line 110 serves to show both the peripheral line of the cannula as well as the circumferential line of the nostril (at least where this line overlaps with the cannula as well as with the nostril wall 102). The latter two lines coincide such that there is a nice fit of the cannula in the nostril. If having such a fit substantially around the complete periphery of the cannula, such that the nostril is in essence completely closed, a very good and reliable administration of the predetermined amount of pharmaceutical composition can be arrived at.

### EXAMPLE 1

When a herd of 960, 3-21 days of age calves has to be treated via intra-nasal administration of a pharmaceutical composition, the veterinarian assembles the required components. In this embodiment, the treatment is a vaccination for respiratory protection of cattle. For this treatment the veterinarian takes 20 boxes of the 50 dose presentations of the Onset™ 5 IN vaccine, a PUMP-IT™ Vaccinator and 50 cannulas that fit this Vaccinator. At the site where the calves are, the vaccine in the first box is reconstituted in BlueShadow Sterile Diluent™ according to manufacturer instructions, and the bottle containing the reconstituted vaccine is connected to the Vaccinator. The veterinarian attaches a bag to his belt, which bag holds the 50 cannulas. A first cannula is taken out of the bag and is slidably connected to the Vaccinator (before or after the bottle containing the vaccine is connected to the Vaccinator). The cannula is inserted into the nasal cavity of a first calf via the corresponding nostril and pushed against the calves nose such that the cannula snuggly fits into the nasal cavity. The Vaccinator is actuated to force a single 2 ml dose of the vaccine into the nasal cavity. Then the vaccinator is retracted such that the cannula leaves the nostril. Then a second calf is vaccinated. After about 25 calves have been vaccinated, the cannula is removed from the Vaccinator and disposed in a cannister attached to the veterinarian's belt. This cannister preferably has a lid with a machined hole that fits the cannula, such that a used cannula can be inserted, but not easily falls out. A new cannula is slidably fitted to it. Alternatively, the cannula is replaced in between every immunological homogenous group (for example in between groups that are kept in the same stable), or even in between every animal treated. The operations are repeated until all calves are vaccinated. Then the used cannulas are removed from the cannister and securely disposed.

1. A method for administering a predetermined amount of a pharmaceutical composition to an animal comprising the steps of:
providing a cannula suitable for insertion into a nasal cavity of the animal,
slidably connecting the cannula to a delivery device suitable for holding the pharmaceutical composition,
inserting the cannula via a nostril into the nasal cavity of the animal such that a peripheral line of the cannula is contiguous with a circumferential line of the nostril, and
actuating the delivery device to force the said amount of the pharmaceutical composition through the cannula into the nasal cavity

2. A method according to claim 1, wherein the delivery device has a hollow protrusion with an outer shape that matches the inner shape of a transport channel of the cannula, which enables sliding the transport channel over the protrusion to form the connection between the cannula and the device.

3. A method according to claim 2, wherein the shapes are substantially circular.

4. A method for preparing a device for administration of a predetermined amount of a pharmaceutical composition to an animal comprising the steps of:
providing a cannula suitable for insertion into a nasal cavity of the animal and being formed such that upon insertion of the cannula via a nostril, a periphery of the cannula is contiguous with a circumference of the nostril,
removably connecting the cannula to a delivery device suitable for holding the pharmaceutical composition by sliding the cannula over a protrusion of the device, thereby forming a channel to transport the composition from the device to the cannula.

5. An apparatus for administering a predetermined amount of a pharmaceutical composition to an animal comprising:
a device with a dispensing mechanism for dispensing the said predetermined amount of the composition,
a space for holding the pharmaceutical composition,
a cannula, slidably connected to the device for delivering the predetermined amount of the composition via the cannula to a nasal cavity of the animal, the cannula being formed such that upon insertion of the cannula via a nostril into the nasal cavity, a peripheral line of the cannula is contiguous with a circumferential line of the nostril.

6. An apparatus according to claim 5, wherein the cannula comprises a cylindrical shaped body having a substantially closed internal channel with an opening for supplying the composition to the channel and a nozzle for delivering the composition into the nasal cavity, the body being provided with a radially extending flange that is inclined with respect to the longitudinal axis of the body.

7. An apparatus according to claim 6, wherein the flange is inclined over an angle that is at least 30°.

8. An apparatus according to claim 7, wherein the angle is from 30° to 60°.

9. An apparatus according to claim 6, wherein the flange is provided with a rim that extends in a plane that is substantially perpendicular to the longitudinal axis of the body.

10. An apparatus according to claim 6, wherein the nozzle is slotted.

11. A cannula as defined in claim 6,.

12. A kit containing a device with a dispensing mechanism for administering a predetermined amount of a pharmaceutical composition to an animal, a container having therein the pharmaceutical composition and multiple cannulas as defined in claim 11.

13. An apparatus according to claim 7, wherein the angle is from 40° to 50°.
